(19) United States
(12) Patent Application Publication
(10) Pub. No.: US 2006/0229675 A1
(43) Pub. Date: Oct. 12, 2006

(54) Anchoring System for Valve Replacement

(75) Inventors: Roberto Novoa, Canton, OH (US); Ben Donaldson, Hatfield, PA (US); Tiffany Yang, Bayside, NY (US); Philip Hum, River Edge, NJ (US)

Correspondence Address: Ben Donaldson, 2296 Rebecca Drive, Hatfield, PA, 19440, US

(73) Assignees: Roberto Novoa, Canton, OH (US); Ben Donaldson, Hatfield, PA (US)

(21) Appl. No.: 11/278,955

(22) Filed: Apr. 6, 2006

Related U.S. Application Data
(60) Provisional application No. 60/594,441, filed on Apr. 7, 2005.

(51) Int. Cl. A61B 17/04

(52) U.S. Cl. 606/232

(57) ABSTRACT

This present invention provides a surgical rivet, a rivet deployment actuator, a rivet cutting instrument and a method for securing a prosthetic valve to the heart which has the potential to substantially decrease the ischemic time required for valve implantation and allow larger prostheses to be inserted in small annuli. The surgical rivet is comprised of two flexible preformed elements capable of assuming a preconfigured shape once it is delivered and secured with the deployment actuator. The deployment actuator is a reusable instrument that lowers the prosthesis to the annulus, secures the prosthesis with an “O” ring and cuts and deploys the flexible elements so that they rest on the sewing ring of the prosthetic valve. The present invention eliminates the tedious and time-consuming tasks of tying sutures which may be particularly difficult in small annuli. It also automatically cuts the sutures to the appropriate length. The use of single rivets allows for expansion to the annulus and insertion of one full size larger (2 mm) prosthesis than what is possible using the classical horizontal pledget mattress technique. This feature is of particular importance in small annuli where prosthesis/patient mismatch can lead to suboptimal long term results. ¹

### FIELD OF INVENTION

This invention relates generally to apparatuses and methods related to cardiac valve replacement. In particular, this invention pertains to a unique surgical fastener and its method of implantation to secure a prosthetic valve to the heart structures in a more efficient and less traumatic way.

### BACKGROUND OF THE INVENTION

When a cardiac valve malfunctions or becomes diseased and is beyond repair, the native valve is replaced with a biological or mechanical prosthesis in order to restore adequate hemodynamics. Traditionally valve replacement operations involve placing the patient on cardiopulmonary bypass, arresting the heart, removing the diseased valve leaflets and securing the prosthetic valve with sutures placed through the valve annulus. After placing the sutures through the annulus and the sewing ring of the prosthesis, the valve is lowered and each of the sutures, which may range from 12 to over 25, has to be individually tied with 4 to 6 surgical knots. This manual process of knotting and securing the sutures is the longest, slowest and perhaps the most difficult portion of valve replacement. It is particularly complicated in small aortic roots where the surgeon's fingers cannot adequately reach.

Adding to the complication is the tension variability that can be applied to the sutures. If the surgeon applies too much tension, the sutures can cut through the tissues or if the surgeon applies too little tension, the sutures are not tied down sufficiently hard resulting in insecure fixations and leaks that require repairs. Thus, in placing each knot the surgeon must use his past experience to judge the optimal tension he applies to the sutures.

Valve replacement surgery is a long, complicated procedure taking approximately 4 hours but even longer when combined with other necessary procedures including coronary bypasses, multi-valve replacement and resection of aneurysms. Although the time required for single valve replacement is well tolerated by patients, when the ischemic time is prolonged, myocardial dysfunction can ensue. ² Additionally, mortality and morbidity increase considerably for lengthy procedures, especially in elderly patients.

Because the classical suturing technique for valve implantation is a time consuming procedure, variations of fasteners have been developed to simplify all types of surgical operations. For cardiac valves in particular, U.S. patent application Ser. No. 09/007,4012 filed Apr. 10, 2002 discloses an annuloplasty, valve repair and valve replacement method employing an unshaped self-closing clip to eliminate the need for suture knotting and suture management. Examples of such clips are disclosed in U.S. patent application Ser. Nos. 09/089,884, 09/090,305, 09/259,705, and 09/260,623 and International Publication Nos. WO 99/12563 and WO 99/62406. While this novel valve replacement method eliminates suture knotting and suture management, reducing surgery time by 38%, all the aforementioned clips have size limitations. ³ Many of the clips are manufactured in various sizes and require the surgeon to predict the right size to use before inserting it into the body. However, before inserting any fastener through the annulus and sewing ring of the prosthesis, the surgeon cannot predict which clip size is needed. The clip size depends not only on the thickness of the annulus but also how the surgeon positions the clip. If the surgeon positions the clip with too much tissue in between the two ends, the clip will not close properly. In this case the surgeon must remove and place a larger one, leading to additional trauma to the tissues and operating room time. Additionally these clips are meant to be used as horizontal mattresses which like the conventional suturing technique, has the tendency to pinch the tissues together and thus decrease the annulus size. In small annuli, this pursestringing effect can lead to insertion of very small prostheses, resulting in patient/prosthesis mismatch and functionally stenotic valves. ⁴

The present invention eliminates the inconveniences associated with the use of traditional suture techniques and currently available fasteners for valve replacement operations.

### SUMMARY OF THE INVENTION

The apparatuses and methods surrounding the present invention will simplify cardiac valve replacement procedure with a more efficient alternative of attaching a prosthetic valve. This invention involves a surgical rivet restrained by a protective sheath which aids in a smoother passage of the rivet through tissues, a deployment actuator that: correctly aligns the rivet with respect to the annulus, causes the rivet to assume its unrestrained position, further secures the rivet with an O ring, cuts the redundant length of rivet and causes the cut end to rest on the sewing ring.

A surgical rivet, according to the present invention, comprises two memory preformed elements encased in a sheath causing the elements to be in a high tension or constrained state, a built-in pledget or stopper at one end and a piercing needle to pass the ensemble through the tissues and sewing ring at the other end. The two preformed elements are comprised of an alloy that is biocompatible and capable of retaining shape memory such as nitinol, although other materials may be used. The shape of the constrained rivet is similar to an inverted “T” whereas the released or unconstrained form resembles an “m” figure. At the point where the two rivet elements connect rests a pledget composed of a type of metal alloy that sits parallel to the annulus. The protective or constraining sheath serves to keep the two elements in a shape that allows them to pass through the tissues and sewing ring smoothly, thereby minimizing trauma. When the elements are released from the protective sheath via a deployment actuator, the elements assume their natural unrestrained position causing apposition of the prosthetic sewing ring and patient's annulus.

In order to pass the rivet through the tissue of the annulus and sewing ring of the prosthesis, the clip is attached to a needle by a long element covered in a protective sheath. In aortic valve replacement, the rivets are placed through the annulus from the ventricular to the aortic side as in the classical suprannular technique. After all the rivets are placed through the annulus, the valve is sized and the needles are then placed through the sewing ring of the prosthetic valve. The valve is then lowered into position. Secure, less traumatic insertion is accomplished by using three appropriately placed deployment actuators without having to use fingers to lower the valve. Once the surgeon feels and sees that the annulus and sewing rings are in good apposition, an “O” ring is lowered and the deployment actuator is activated to strip the protective sheath and allows the elements to unfold onto the sewing ring in its natural unrestrained position. Upon activation, the actuator also flattens the “O” ring to secure the two elements in place, forcing the flexible elements to rest on the sewing ring.

Should the need arise to remove the rivet, a specially designed tool is inserted between the annulus and sewing ring of the prosthesis.

### BRIEF DESCRIPTION OF THE DRAWINGS

The following detailed descriptions in conjunction with the accompanying drawings will illustrate the advantages and provide a better understanding of the present invention.

FIGS. 1A-1B are variations of views of a restrained surgical fastener according to the present invention, where FIG. 1A shows the front view of the restrained surgical fastener, and FIG. 1B shows the side view of the restrained surgical fastener.

FIG. 2A-2B are perspective views of the cross-section of the wire that compose the rivet where 2A is circular and 2B is non-circular.

FIG. 3A-3B are variations of views of the permanently attached pledget to the preformed elements, where FIG. 3A shows the front view, FIG. 3B shows the side view.

FIG. 4 illustrates how the configuration of the built-in pledget in conjunction with the cross-sectional shape of the preformed elements forces the correct alignment of the rivet to the longitudinal axis of the annulus.

FIG. 5A-5B shows the preformed elements absent of the protective sheath in its natural unrestrained “m” configuration.

FIG. 6 is a view of the instrument used on the fastener.

FIG. 7 is a view of the “O” ring spirally coiled like a spring.

FIGS. 8A-8E are variations of views of the deployment actuator used to fix the “O” ring around the preformed elements to lock in the annulus and prosthesis between the “O” ring and the pledget, where FIG. 8A is a schematic view of the actuator with an undeployed “O” ring, FIG. 8B is a sectional view of FIG. 8A, FIG. 8C is a view of the actuator in the process of deploying the “O” ring, FIG. 8D is a sectional view of FIG. 8C to show the protective sheath being stripped off the preformed elements when the inner tube is removed after the “O” ring is deployed.

FIG. 9A-9E illustrate the valve replacement procedure of the present invention, where FIG. 9A shows the insertion of the rivet, FIG. 9B shows an alternative to single, separate fasteners, FIG. 9C show the protective sheath threaded through the inner tube, FIG. 9D shows the tightening of the connection between the prosthesis and annulus, FIG. 9E shows the unrestrained rivet holding the sewing ring and annulus together.

### DETAILED DESCRIPTION OF THE INVENTION

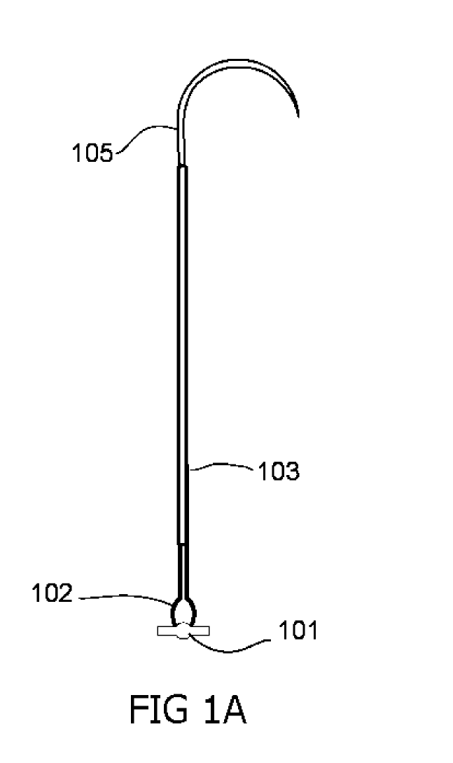

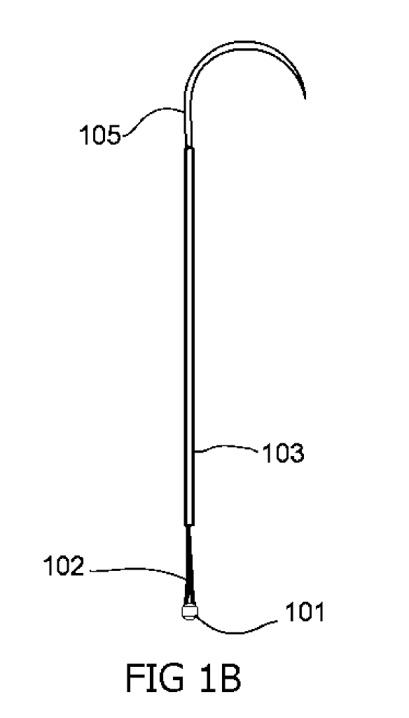

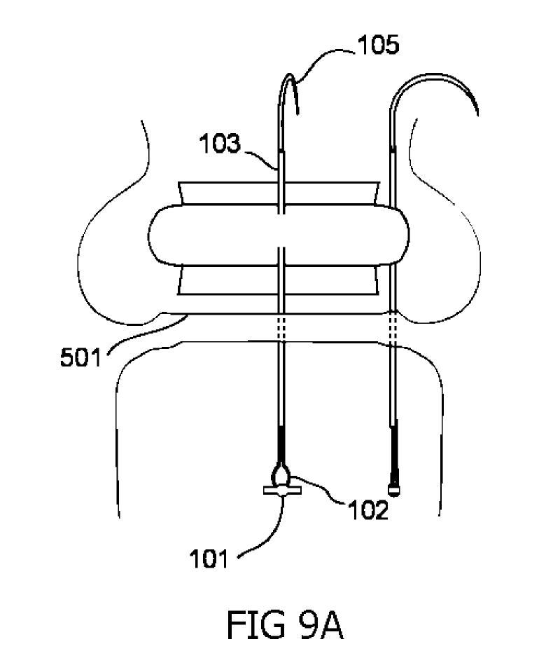

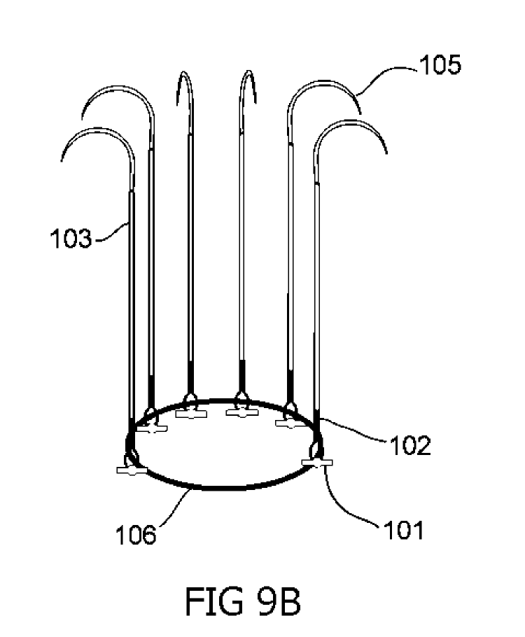

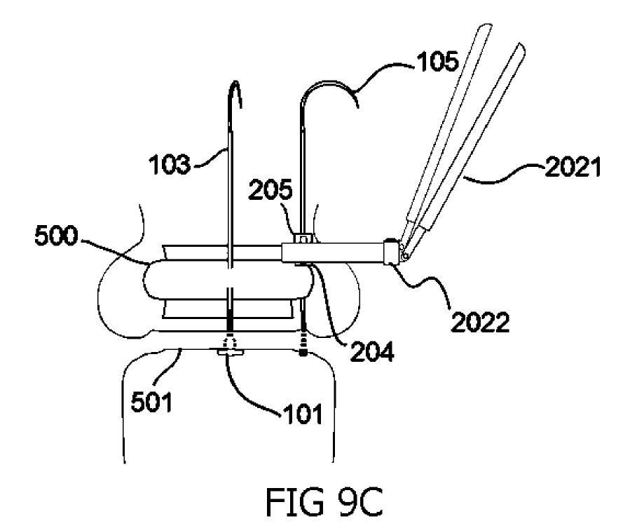

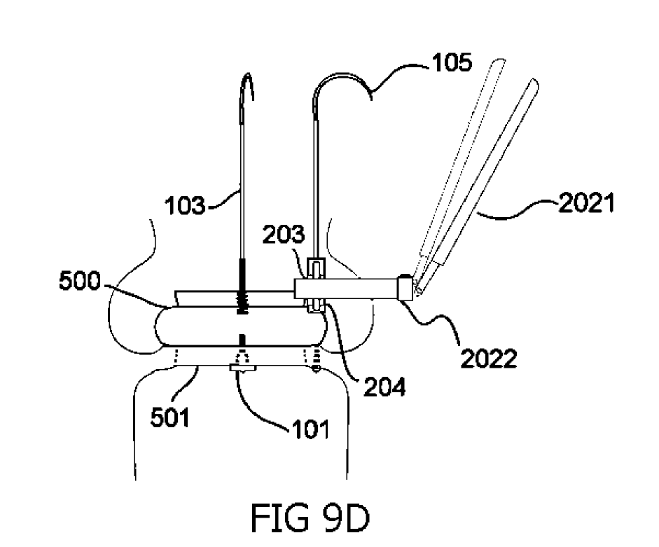

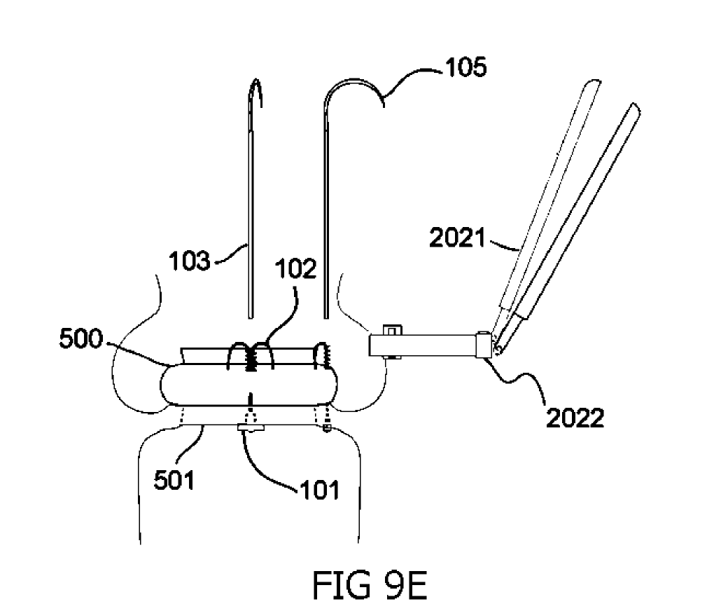

This present invention relates to apparatuses and methods for simplifying cardiac valve replacement involving a unique surgical rivet 100 shown schematically in FIGS. 1A-1B, used to attach a prosthesis sewing ring to a valvular annulus shown in FIG. 9A. The following description in conjunction with the drawings explains the details of the invention.

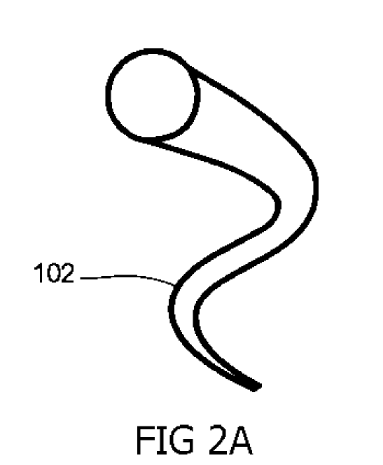

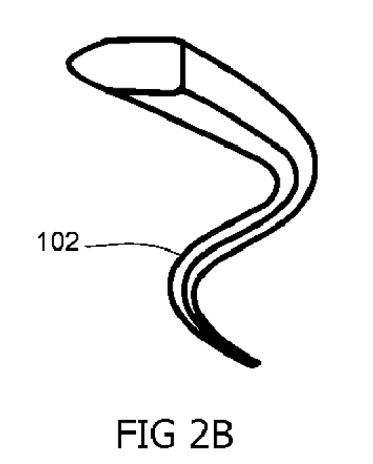

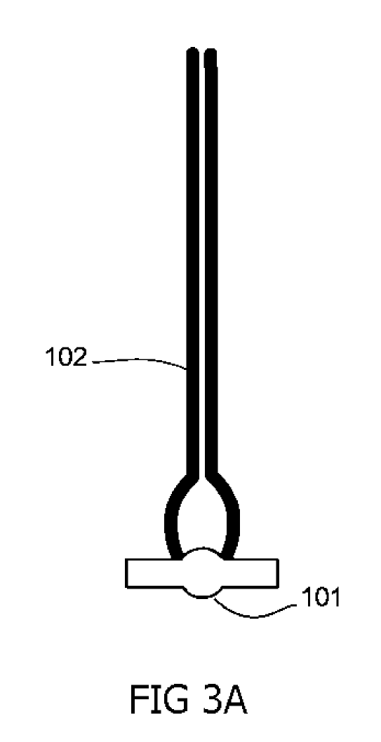

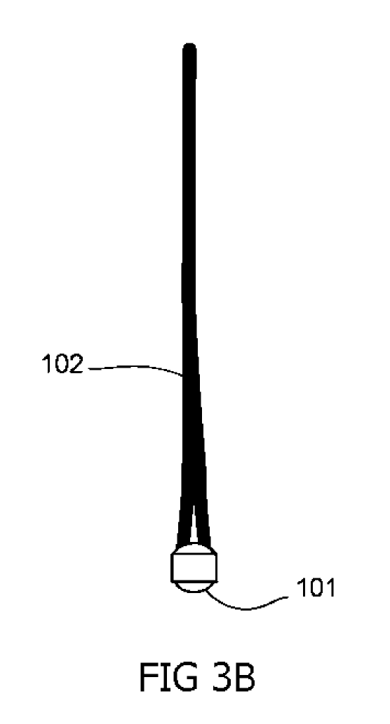

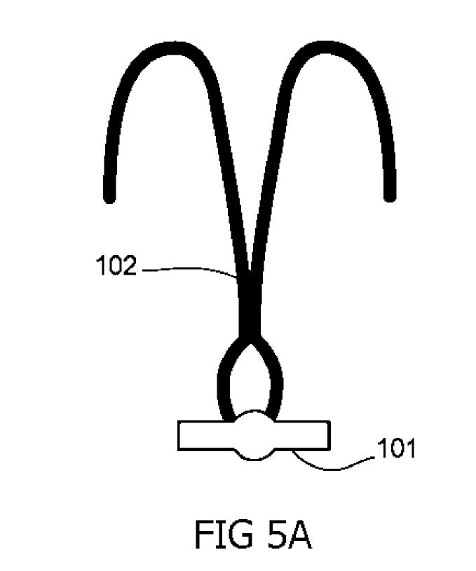

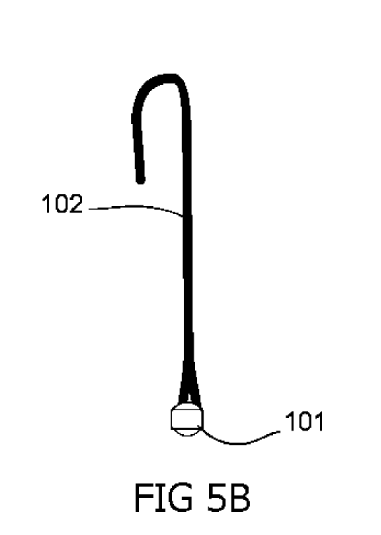

Referring to the drawings, and first to FIG. 1A, is the surgical rivet 100 comprise of a metallic pledget or stopper 101, two memory alloy preformed elements 102 encased in a protective sheath 103 causing the elements to be in a high tension or constrained state, attaching the memory alloy elements to a surgical needle 105. The two preformed elements 102 are wire composed of nitinol or some other memory alloy that can have a circular cross-section shown in FIG. 2A or non-circular cross-section shown in FIG. 2B to resist rotation about the elongated direction, permitting the fastener to also resist rotation. The two preformed elements 102, connected by a metallic pledget 101 as seen in FIG. 3A-3B, are movable between a restrained position (FIGS. 1A-1B) and unrestrained position (FIG. 5).

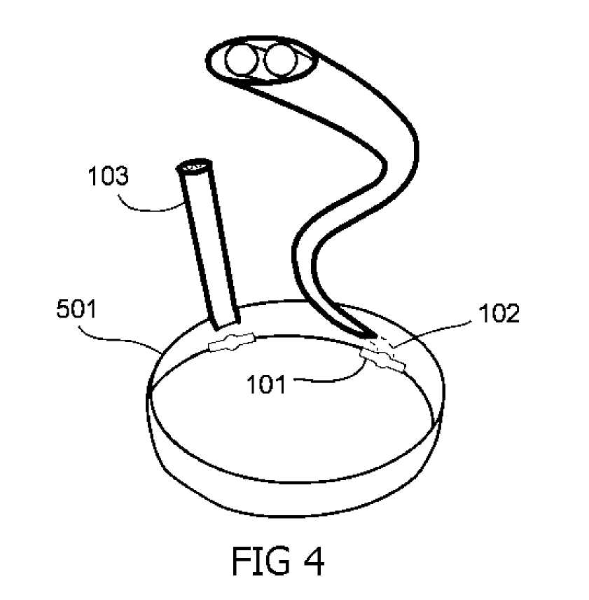

The pledget 101 composed of a metallic material is permanently attached to the preformed elements 102 by welding or any other suitable means of connection. FIG. 4 shows how the rectangular shape of the pledget 101 makes it natural for the pledget 101 to align its lengthier side parallel to the annulus 501. However because of the rotational symmetry of the currently used sutures, the pledget 101 has a tendency to torque out of place at the time of suture placement. But, because the cross-sectional shape of the preformed elements 102 of the present invention is non-circular, which eliminates the rotational symmetry, the pledget 101 is held in place and does not torque. Thus, the configuration of the built-in pledget in conjunction with the shape of the preformed elements 102 forces the correct alignment of the rivet 100 to the longitudinal axis of the annulus 501 as illustrated by FIG. 4.

In the pre-deployment state (FIGS. 1A-1B), the preformed elements 102 are parallel and held in tension in an inverted “T” shape by the protective sheath 103. The protective sheath 103 is essentially a hollow elliptical cylinder that fits snugly around the preformed elements 102 and can slide over the preformed element. In the deployed state shown in FIG. 5, the elements 102, absent of the protective sheath 103, are free and assume its natural unrestrained configuration where the ends of the elements are preformed to curl assuming an “m” shape.

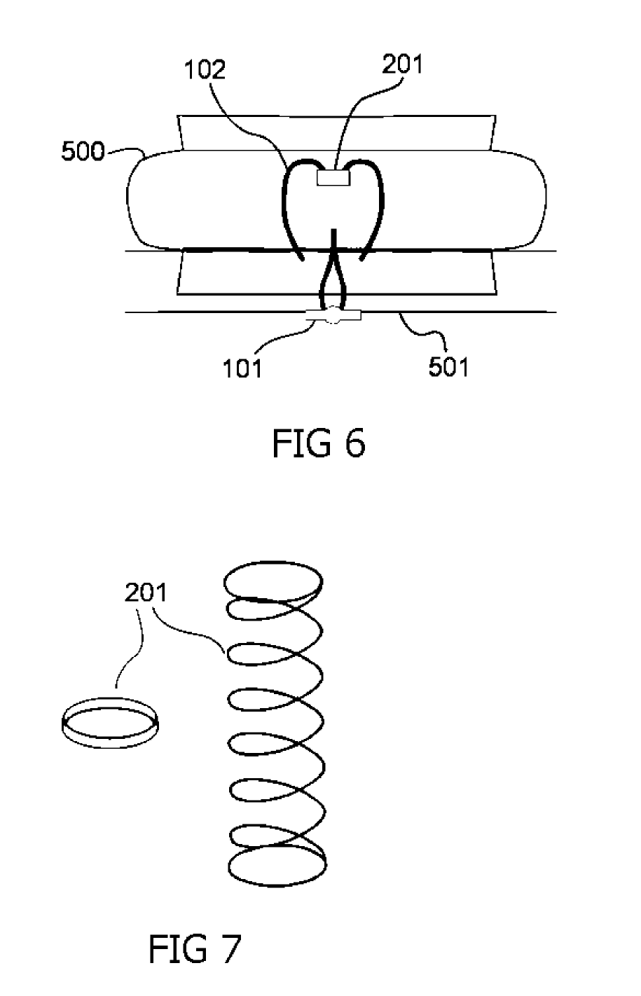

FIG. 6 illustrates an “O” ring 201 holding the preformed elements 102 together and securing the connection between a prosthetic valve 500 and annulus 501. The “O” ring 201 is spirally coiled like a spring shown in FIG. 7 or some other configuration that serves the same function. When the ends of the preformed elements 102 are free of the protective sheath 103, the ends curl in to assume the “m” shape which applies tension to the annulus/prosthesis junction. Said tension is further reinforced by the “O” ring 201 which further secures it in place. Since the shape of the “O” ring 201 is specially designed to apply a reaction force, it springs back against the ends of the preformed elements so there will always be the same constant net force sandwiching the valve 500 and annulus 501 between the “O” ring 201 and the pledget 101. This eliminates the variability in tension associated with the current conventional suture technique as mentioned previously.

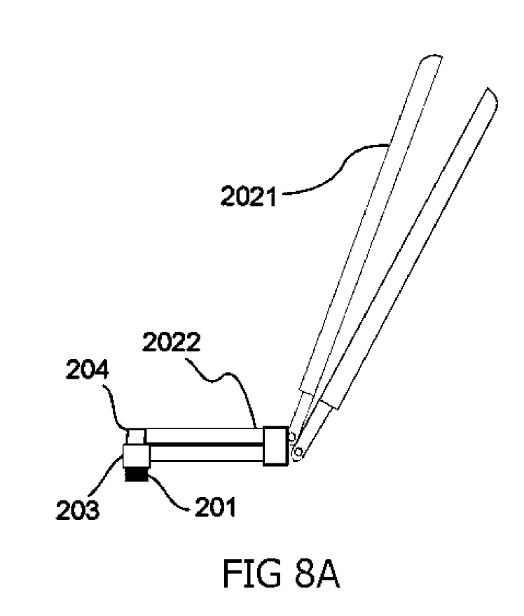

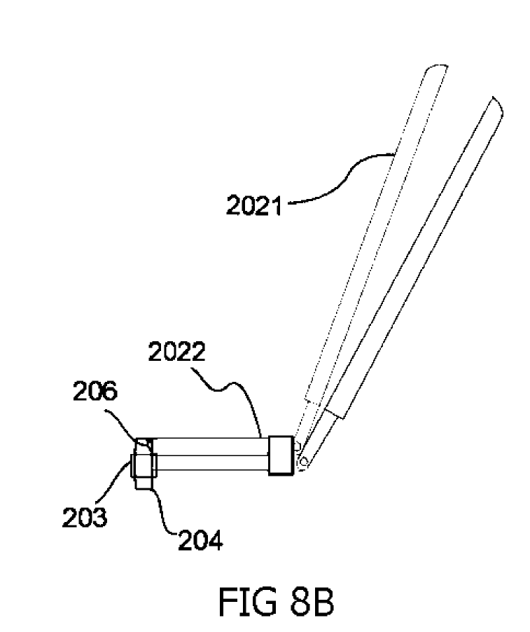

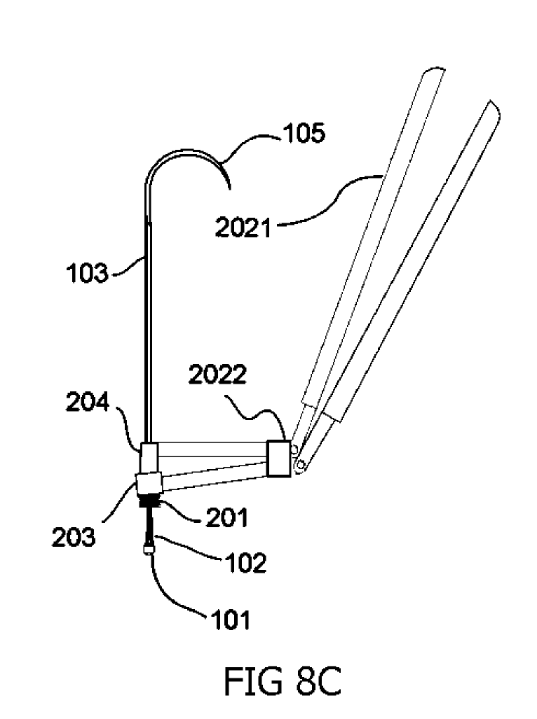

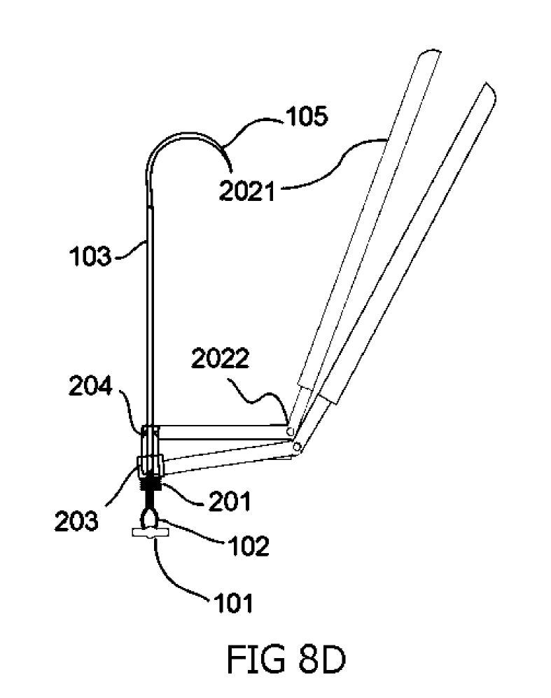

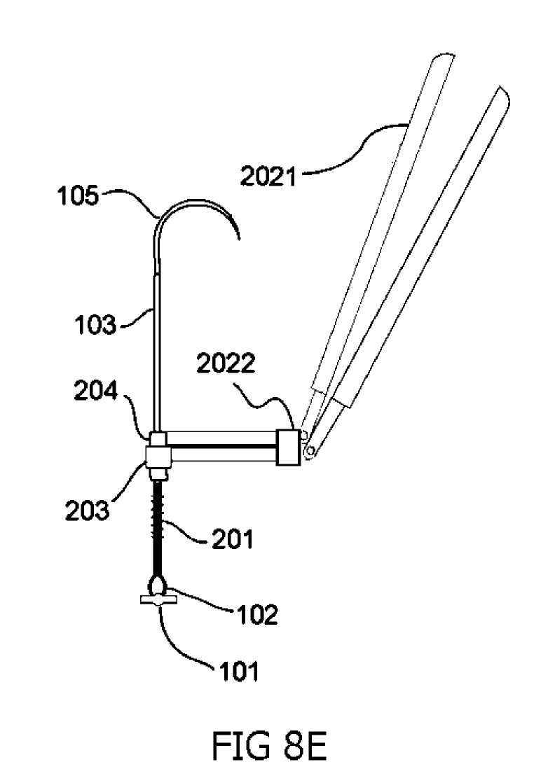

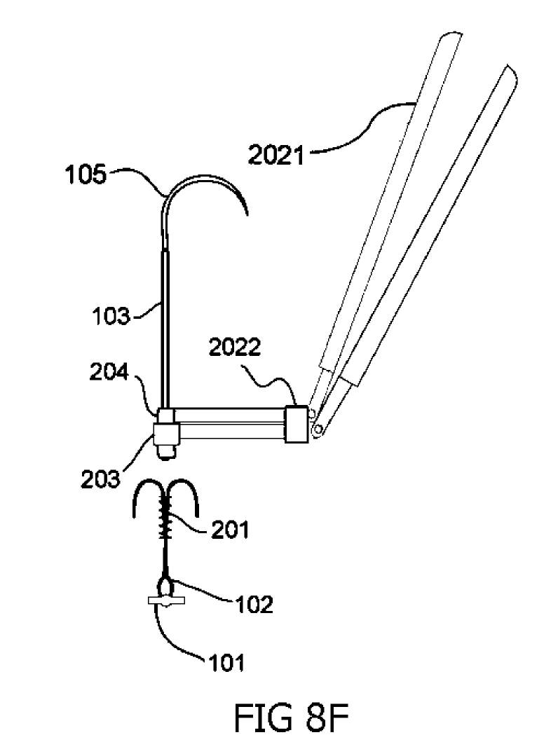

Included in the present invention is the deployment actuator 200 of FIG. 8A and FIG. 8C used to deploy an “O” ring 201 and strip the protective sheath 103 off the elements 102. The deployment actuator 200 comprises of an elongated shaft assembly 202 having a distal end 2021 and a proximal end 2022 with an outer tube 203 that can easily slide over a hollow inner tube 204. The inner tube 204 is longer than the outer tube 203 and has a slit 205 down the middle for the protective sheath 103 to pass through. At the distal end 2021 portion of the inner tube 204, which is slightly larger than the opening of the “O” ring 201, the inner tube 204 holds the “O” ring 201 in radial tension. To deploy the “O” ring 201 the outer tube 203 slides over the inner tube 204, pushing the “O” ring 201 off the inner tube 204 and transferring it onto the preformed elements 102.

Also at the distal end portion 2021 but on the inside of the inner tube 204 is a mechanism 206 (FIG. 8B) that strips the protective sheath 103 off the preformed elements 102. This stripper mechanism 206 is constructed in such a way that when the protective sheath 103 slides into the mechanism 206 nothing is affected. But when the protective sheath 103 slides out, the mechanism 206 is activated and the protective sheath 103 is stripped off the preformed elements 102. This occurs simultaneously (FIG. 8D) when the inner tube 204 is removed after the “O” ring 201 is deployed. Once the elements 102 are free of the protective sheath 103 they are no longer constrained and can assume their natural unrestrained “m” configuration (FIG. 8F).

The valve replacement procedure visualized in FIGS. 9A-9E embodying this invention includes a surgical rivet 100 comprised of two memory alloy, preformed elements 102 encased in a protective sheath 103 causing the elements 102 to be in a high tension or constrained state, a pledget 101 or stopper at one end and a piercing needle 105 to pass through the tissues at the opposite end. All methods prior to and including trimming of the annular leaflets follow the current standard valve replacement surgical procedure. After the leaflets are trimmed, a surgical rivet 100 is inserted into the annulus 501 using the piercing needle 105 to puncture the annulus 501 and to pull the surgical rivet 100 through until the pledget 101 reaches the annulus (FIG. 9A). The appropriate number of rivets is inserted in the same fashion and the valve is sized. Alternative, the rivets 100 can be connected by long strands of flexible wire or other material 106 made of a type of biocompatible material as in FIG. 9B. The surgeon can choose the appropriate number of rivets 100 by cutting off the excess after the rivets 100 are inserted into the annulus 501. The protective sheath 103 of the rivet 100 is threaded through the inner tube 204 and exits from the slit 205 shown in FIG. 9C. FIG. 9D illustrates how the connection between the prosthetic valve 500 and annulus 501 is tightened. With one hand the surgeon pushes the inner tube 204 down on the sewing ring 500 and with the other hand the surgeon pulls the protective sheath 103 in the opposite direction. The outer tube 203 is slid over the inner tube 204 to transfer the “O” ring 201 to the preformed elements 102. While the outer tube 203 is held against the sewing ring 500, the inner tube 204 is removed to strip the protective sheath 103 off the preformed elements 102. FIG. 9E shows the elements 102 in its unrestrained “m” configuration sandwiching the prosthesis 500 and annulus 501 between the pledget 101 and the unrestrained preformed elements 102. In the same picture, notice that the inverted “T” shape of the preformed elements 102 does not pinch the fibers together as sutures and other fasteners do, and thus does not decrease the native valve size.

The description and examples are not meant to limit the invention. There can be variations as long as the functions remain within the details of the invention.

References Cited - US Patent Documents

| | | | |
|---|---|---|---|
| 43,098 | A | June 1864 | Cooper |
| 655,190 | A | August 1900 | Bramson |
| 1,583,271 | A | May 1926 | Biro |
| 1,625,602 | A | April 1927 | Gould et al. |
| 2,240,330 | A | April 1941 | Flagg et al. |
| 2,256,382 | A | September 1941 | Dole |
| 2,264,679 | A | December 1941 | Ravel |
| 2,516,710 | A | July 1950 | Mascolo |
| 2,715,486 | A | August 1955 | Marcoff-Moghadam et al |
| 2,890,519 | A | June 1959 | Storz, Jr. |
| 3,249,104 | A | May 1966 | Hohnstein |
| 3,274,658 | A | September 1966 | Pile |
| 3,452,742 | A | July 1969 | Muller |
| 3,506,012 | A | April 1970 | Brown |
| 3,509,882 | A | May 1970 | Blake |
| 3,547,103 | A | December 1970 | Cook |
| 3,570,497 | A | March 1971 | Lemole |
| 3,608,095 | A | September 1971 | Barry |
| 3,620,212 | | November 1971 | Fannon et al. |
| 3,638,654 | A | February 1972 | Akuba |
| RE27,391 | E | June 1972 | Merser |
| 3,753,438 | A | August 1973 | Wood et al. |
| 3,786,806 | | January 1974 | Johnson et al. |
| 3,875,648 | A | April 1975 | Bone |
| 3,890,977 | | June 1975 | Wilson |
| 3,910,281 | A | October 1975 | Kletschka et al. |
| 3,958,576 | A | May 1976 | Komiya |
| 4,038,725 | A | August 1977 | Keefe |
| 4,103,690 | A | August 1978 | Harris |
| 4,140,125 | A | February 1979 | Smith |
| 4,170,990 | A | October 1979 | Baumgart et al. |
| 4,192,315 | A | March 1980 | Hilzinger et al. |
| 4,217,902 | A | August 1980 | March |
| 4,324,248 | A | April 1982 | Perlin |
| 4,345,601 | A | August 1982 | Fukuda |
| 4,416,266 | A | November 1983 | Baucom |
| 4,456,017 | A | June 1984 | Miles |
| 4,485,816 | A | December 1984 | Krumme |
| 4,522,207 | A | June 1985 | Klieman et al. |
| 4,535,764 | A | August 1985 | Ebert |
| 4,549,545 | A | October 1985 | Levy |
| 4,586,502 | A | May 1986 | Bedi et al. |
| 4,586,503 | A | May 1986 | Kirsch et al. |
| 4,595,007 | A | June 1986 | Mericle |
| 4,612,932 | A | September 1986 | Caspar et al. |
| 4,637,380 | A | January 1987 | Orejola |
| 4,665,906 | A | May 1987 | Jervis |
| 4,683,895 | A | August 1987 | Pohndorf |
| 4,719,924 | A | January 1988 | Crittenden et al. |
| 4,730,615 | A | March 1988 | Sutherland et al. |
| 4,809,695 | A | March 1989 | Gwathmey et al. |
| 4,873,975 | A | October 1989 | Walsh et al. |
| 4,896,668 | A | January 1990 | Popoff et al. |
| 4,899,744 | A | February 1990 | Fujitsuka et al. |
| 4,901,721 | A | February 1990 | Hakki |
| 4,924,866 | A | May 1990 | Yoon |
| 4,926,860 | A | May 1990 | Stice et al. |
| 4,929,240 | A | May 1990 | Kirsch et al. |
| 4,932,955 | A | June 1990 | Merz et al. |
| 4,950,283 | A | August 1990 | Dzubow et al. |
| 4,950,285 | A | August 1990 | Wilk |
| 4,983,176 | A | January 1991 | Cushman et al. |
| 4,990,152 | A | February 1991 | Yoon |
| 4,997,439 | A | March 1991 | Chen |
| 5,002,550 | A | March 1991 | Li |
| 5,002,562 | A | March 1991 | Oberlander |
| *5,002,563 | A | March 1991 | Pyka et al. |
| 5,026,379 | | June 1991 | Yoon |
| 5,047,047 | A | September 1991 | Yoon |
| 5,053,047 | A | October 1991 | Yoon |
| 5,074,874 | A | December 1991 | Yoon et al. |
| 5,100,418 | A | March 1992 | Yoon et al. |
| 5,123,913 | A | June 1992 | Wilk et al. |
| 5,152,769 | A | October 1992 | Baber |
| 5,154,189 | A | October 1992 | Oberlander |
| 5,158,566 | A | October 1992 | Pianetti |
| 5,171,250 | A | December 1992 | Yoon |
| 5,171,252 | A | December 1992 | Friedland |
| 5,174,087 | A | December 1992 | Bruno |
| 5,196,022 | A | March 1993 | Bilweis |
| 5,219,358 | A | June 1993 | Bendel et al. |
| 5,222,976 | A | June 1993 | Yoon |
| 5,236,440 | A | August 1993 | Hlavacek |
| 5,242,456 | A | September 1993 | Nash et al. |
| 5,246,443 | A | September 1993 | Mai |
| 5,258,011 | A | November 1993 | Drews |
| 5,269,783 | A | December 1993 | Sander |
| 5,209,289 | A | March 1994 | Sanders et al. |
| 5,304,204 | A | April 1994 | Bregen |
| 5,312,436 | A | May 1994 | Coffey et al. |
| 5,330,503 | A | July 1994 | Yoon |
| 5,336,239 | A | August 1994 | Gimpelson |
| 5,345,424 | A | October 1994 | Buzerak et al. |
| 5,374,268 | A | December 1994 | Sander |
| 5,383,904 | A | January 1995 | Totakura et al. |
| 5,403,346 | A | April 1995 | Loeser |
| 5,437,680 | A | August 1995 | Yoon |
| 5,437,685 | A | August 1995 | Blasnik |
| 5,439,479 | A | August 1995 | Shichman et al. |
| 5,445,167 | A | August 1995 | Yoon et al. |
| 5,450,860 | A | September 1995 | O'Connor |
| 5,452,733 | A | September 1995 | Sterman et al. |
| 5,456,246 | A | October 1995 | Schmieding et al. |
| 5,462,561 | A | October 1995 | Voda |
| 5,474,557 | A | December 1995 | Mai |
| 5,480,405 | A | January 1996 | Yoon |
| 5,486,187 | A | January 1996 | Le et al. |
| 5,499,990 | A | March 1996 | Schulken et al. |
| 5,500,000 | A | March 1996 | Faegin et al. |
| 5,527,342 | A | June 1996 | Pietrzak et al. |
| 5,549,619 | A | August 1996 | Peters et al. |
| 5,569,274 | A | October 1996 | Rapacki et al. |
| 5,569,301 | A | October 1996 | Granger et al. |
| 5,582,616 | A | December 1996 | Bolduc et al. |
| 5,582,619 | A | December 1996 | Ken |
| 5,586,983 | A | December 1996 | Sanders et al. |
| 5,591,179 | A | January 1997 | Edelstein |
| 5,593,414 | A | January 1997 | Shipp et al. |
| 5,593,424 | A | January 1997 | Northrup, III |
| 5,609,608 | A | March 1997 | Benett et al. |
| 5,632,752 | A | May 1997 | Buelna |
| 5,632,753 | A | May 1997 | Loeser |
| 5,643,295 | A | July 1997 | Yoon |
| 5,645,568 | A | July 1997 | Chervitz et al. |
| 5,665,109 | A | September 1997 | Yoon |
| 5,683,417 | A | November 1997 | Cooper |
| 5,695,505 | A | December 1997 | Yoon |
| 5,697,943 | A | December 1997 | Sauer et al. |
| 5,700,270 | A | December 1997 | Peyser et al. |
| 5,700,271 | A | December 1997 | Whitfield et al. |
| 5,707,380 | A | January 1998 | Hinchliffe et al. |
| 5,709,693 | A | January 1998 | Taylor |
| 5,709,695 | A | January 1998 | Northrup, III |
| 5,725,539 | A | March 1998 | Matern |
| 5,725,542 | A | March 1998 | Yoon |
| 5,728,135 | A | March 1998 | Bregen et al. |
| 5,735,290 | A | April 1998 | Sterman et al. |
| 5,799,661 | A | September 1998 | Boyd et al. |
| 5,810,851 | A | September 1998 | Yoon |
| 5,810,882 | A | September 1998 | Bolduc et al. |
| 5,820,631 | A | October 1998 | Nobles |
| 5,824,008 | A | October 1998 | Bolduc et al. |
| 5,830,221 | A | November 1998 | Stein et al. |
| 5,849,019 | A | December 1998 | Yoon |
| 5,879,371 | A | March 1999 | Gardiner et al. |
| 5,891,130 | A | April 1999 | Palermo et al. |
| 5,891,160 | A | April 1999 | Williamson, IV et al. |
| 5,895,394 | A | April 1999 | Kienzle et al. |
| 5,961,539 | A | October 1999 | Northrup, III et al |
| 5,964,772 | A | October 1999 | Bolduc et al. |
| 5,972,024 | A | October 1999 | Northrup, III et al. |
| 5,976,159 | A | November 1999 | Bolduc et al. |
| 5,984,917 | A | November 1999 | Fleischman et al. |
| 5,989,242 | A | November 1999 | Saadat et al. |
| 5,989,268 | A | November 1999 | Pugsley, Jr. et al. |
| 5,997,556 | A | December 1999 | Tanner |
| 6,001,110 | A | December 1999 | Adams |
| 6,013,084 | A | January 2000 | Ken et al. |
| *6,074,401 | A | June 2000 | Gardiner et al. |
| 6,132,438 | A | October 2000 | Fleischman et al. |
| 6,139,540 | A | October 2000 | Rost et al. |
| 6,143,004 | A | November 2000 | Davis et al. |
| *6,149,658 | A | November 2000 | Gardiner et al. |
| 6,176,413 | B1 | January 2001 | Heck et al. |
| 6,190,373 | A | February 2001 | Palermo et al. |
| 6,193,733 | B1 | February 2001 | Adams |
| 6,193,734 | A | February 2001 | Bolduc et al. |
| 6,254,625 | B1 | July 2001 | Bolduc et al. |
| 6,296,656 | A | October 2001 | Bolduc et al. |
| 6,346,112 | A | February 2002 | Adams |
| *6,551,332 | B1 | April 2003 | Nguyen et al. |
| *6,607,541 | B1 | September 2003 | Gardiner et al. |
| *6,613,059 | B2 | September 2003 | Schaller et al. |
| *6,641,593 | B1 | November 2003 | Schaller et al. |

References Cited - Foreign Patent Documents

| | | |
|---|---|---|
| EP | 0 121 362 A1 | October 1984 |
| EP | 0 140 557 A2 | May 1985 |
| EP | 0 326 426 B1 | August 1989 |
| EP | 0 419 597 B1 | April 1991 |
| EP | 0 432 692 A1 | June 1991 |
| EP | 0 478 949 B1 | April 1992 |
| EP | 0 494 636 A1 | July 1992 |
| EP | 0 559 429 A1 | September 1993 |
| EP | 0 641 546 A1 | March 1995 |
| EP | 0 711 532 A1 | May 1996 |
| EP | 0 734 697 A2 A3 | October 1996 |
| EP | 0 537 955 B1 | December 1996 |
| EP | 0 778 005 A1 | June 1997 |
| EP | 0 815 795 A1 | January 1998 |
| GB | 2 223 410 A | April 1990 |
| WO | WO 90/06725 A1 | June 1990 |
| WO | WO 90/09149 A1 | August 1990 |
| WO | WO 90/14795 A2 | December 1990 |
| WO | WO 91/07916 A1 | June 1991 |
| WO | WO 91/17712 A1 | November 1991 |
| WO | WO 92/05828 A1 | April 1992 |
| WO | WO 94/15535 A1 | July 1994 |
| WO | WO 94/15537 A1 | July 1994 |
| WO | WO 96/00035 A1 | January 1996 |
| WO | WO 96/06565 A1 | March 1996 |
| WO | WO 96/38090 A1 | December 1996 |
| WO | WO 97/28744 A1 | August 1997 |
| WO | *WO 97/32526 A1 | September 1997 |
| WO | WO 97/42881 A1 | November 1997 |
| WO | *WO 98/30153 A1 | July 1998 |
| WO | WO 98/42262 A1 | October 1998 |
| WO | *WO 99/62406 A2 | December 1999 |
| WO | *WO 99/62409 A1 | December 1999 |
| WO | WO 01/17441 | April 2001 |
| WO | WO 02/30295 A1 | April 2002 |
| WO | *WO 02/87425 A2 | May 2002 |
| WO | WO 03/88875 A1 | October 2003 |

(1) Pibarot P, et al. Hemodynamic and clinical impact of prosthesis-patient mismatch in the aortic valve position and its prevention. J Am Coll Cardiol. 2000 October; 36(4): 1131-41.
(2) Khudairi T, et al. Preservation of ischemic myocardial function and integrity with targeted cytoskeleton-specific immunoliposomes. J Am Coll Cardiol. 2004; 43(9): 1683-9.
(3) Kypson A P, et al. Robotic Cardiac Surgery. J of Long-Term Effects of Medical Implants. 2003; 13(6): 451-464.
(4) Gonzalez-Juanatey J R, et al. Influence of the size of aortic valve prostheses on hemodynamics and change in left ventricular mass: implications for the surgical management of aortic stenosis. J Thorac Cardiovasc Surg. 1996 August; 112(2): 273-80.

What is claimed is:

1. A surgical rivet comprising two memory alloy, preformed elements movable between a constrained configuration and an unconstrained configuration, a permanently attached pledget or stopper, and a piercing needle.

2. The elements as in claim 1, wherein said elements are encased in a protective sheath for improving the passage through tissues.

3. The surgical rivet of claim 1, wherein said rivet self-aligns parallel to the longitudinal axis of the annulus.

4. The surgical rivet of claim 1, wherein said elements penetrate the same incision.

5. The surgical rivet of claim 1, wherein the cross-section of each said element is of a non-circular shape to prevent rotation about its longitudinal axis.

6. The surgical rivet of claim 1, wherein the length of said rivet elements can be adjusted depending on the thickness of the tissue.

7. The surgical rivet of claim 1, wherein said elements is an inverted “T” in its restrained configuration.

8. The surgical rivet of claim 1, wherein said elements coil into its unrestrained preformed “m” configuration.

9. The surgical rivet of claim 1, wherein said rivets can be connected by a flexible wire.

10. The surgical rivet of claim 9, wherein said flexible wire has the length of the circumference of the largest possible prosthesis.

11. The surgical rivet of claim 1, wherein said pledget is composed of a metallic alloy.

12. A deployment actuator for a surgical rivet comprising: an elongated shaft assembly having at the distal end portion a mechanism deploying an “O” ring; a stripping mechanism to remove the protective sheath; a cutting element to cut the elements to the appropriate length; and preformed grooves to allow the elements to fold on to the sewing ring.

13. The deployment actuator of claim 12, wherein said “O” ring prevents the creation of a larger hole in the sewing ring of the prosthesis.

14. The deployment actuator of claim 12, wherein said “O” ring secures said rivet of claim 1 in place when in its unrestrained position.

15. The deployment actuator of claim 12, wherein said “O” ring is spirally twisted in a spring-like figure.

16. An instrument for removing a surgical rivet comprising of a flat double action cutter made of high grade titanium or of similarly strong metallic alloy.

17. A cardiac valve replacement method comprising the following steps:
providing a self-gauging surgical fastener, comprising of a rivet with two elements movable between a constrained configuration and an unconstrained configuration and a built-in metallic pledget; providing a deployment actuator with an elongated shaft assembly having at the distal end portion, a mechanism for displacing the “O” ring, a stripping mechanism to remove the protective sheath, a cutting element to trim the elements to the appropriate length, and preformed grooves to allow the elements to fold on to the sewing ring; and
providing a removal instrument comprised of a flat double action cutter made of high grade titanium or of a similarly strong metallic alloy.

18. The replacement method of claim 17 further comprising the steps of:
placing said surgical fasteners radially around the annulus in their restrained position;
advancing the needle attached to said surgical rivet through the prosthesis sewing ring and annulus; and using single or multiple said deployment actuators appropriately placed allowing said fasteners to assume their natural unrestrained configuration and displacing said “O” ring.
